(19) United States
(12) Patent Application Publication
(10) Pub. No.: US 2022/0143161 A1
(43) Pub. Date: May 12, 2022

(54) MRNA VACCINE

(71) Applicant: eTheRNA immunotherapies NV, Niel (BE)

(72) Inventors: Stefaan De Koker, Niel (BE); Lukasz Bialkowski, Niel (BE)

(21) Appl. No.: 17/435,561

(22) PCT Filed: Mar. 13, 2020
(86) PCT No.: PCT/EP2020/056891

§ 371 Date: Sep. 1, 2021

(30) Foreign Application Priority Data
Mar. 13, 2019 (EP) 19162558.1

(51) Int. Cl. A61K 39/00; A61K 39/39; A61K 39/395; A61P 35/00

(52) U.S. Cl. CPC A61K 39/0011; A61K 39/39; A61K 39/3955; A61K 2039/55516; A61K 2039/53; A61K 2039/55561; A61P 35/00

(57) ABSTRACT

The present invention in general relates to a combination of mRNA molecules encoding functional immunostimulatory proteins and a CTLA4 pathway inhibitor. In particular, it relates to a combination of one or more mRNA molecules encoding at least one functional immunostimulatory protein selected from the list comprising: CD40L, CD70 and caTLR4; and a CTLA4 pathway inhibitor, optionally also in the form of an mRNA molecule. The present invention further relates to vaccines comprising such combination, as well as uses of the combinations and vaccine of the present invention in human or veterinary medicine, in particular in the prevention and/or treatment of cell proliferative disorders.

### FIELD OF THE INVENTION

The present invention in general relates to a combination of mRNA molecules encoding functional immunostimulatory proteins and a CTLA4 pathway inhibitor. In particular, it relates to a combination of one or more mRNA molecules encoding at least one functional immunostimulatory protein selected from the list comprising: CD40L, CD70 and caTLR4; and a CTLA4 pathway inhibitor, optionally also in the form of an mRNA molecule. The present invention further relates to vaccines comprising such combination, as well as uses of the combinations and vaccine of the present invention in human or veterinary medicine, in particular in the prevention and/or treatment of cell proliferative disorders.

### BACKGROUND TO THE INVENTION

The induction of potent cytolytic CD8 T cell responses capable of recognizing and killing cancer cells constitutes the key goal of any therapeutic cancer vaccine. The capacity of a vaccine to elicit such cytolytic T cell responses is heavily determined by the early interaction between the vaccine and dendritic cells (DCs), the most potent antigen presenting cells and instigators of T cell immunity. In contrast to protein-based vaccines, mRNA vaccines enable expression of the mRNA encoded antigen in the cytosol of DCs, the natural route of antigen processing and presentation of antigens to CD8 T cells.

In addition, in vitro transcribed mRNA—produced by viral polymerases such as T7—partially resembles a viral RNA, and is hence recognized by innate immune sensors, endowing the mRNA with intrinsic adjuvant properties (Kariko et al., 2005; Yoneyama et al., 2010). Nonetheless, activation of DCs by IVT mRNA is suboptimal, and can be further enhanced by the co-delivery of TriMix mRNA, a mix of three mRNAs encoding the immune-stimulatory proteins CD40L, CD70 and caTLR4 (Bonehill et al., 2008; Van Lint et al. 2012; Van Lint et al., 2016). Addition of TriMix mRNA to mRNA encoding tumor antigens has been demonstrated to strongly enhance the magnitude of the T cell response and its antitumor efficacy in preclinical models and is currently explored in clinical studies.

Anti-CTLA4 antibodies can interfere with tumor immunosuppression and restore the antitumor efficacy of pre-existing effector T cells, with the anti-CTLA-4 blocking antibody ipilimumab being the first immune checkpoint inhibitor to be approved for the treatment of cancer patients (Seidel et al., 2018). Interaction of CTLA-4 with CD80 and CD86 present on the surface of antigen presenting cells provides an inhibitory signal to T cells during initial priming. In addition, CTLA-4 is highly expressed at the surface of regulatory T cells. mRNA vaccines have the capacity to elicit/expand anti-tumor T cells directed against the mRNA encoded tumor-associated antigen. Nonetheless, therapeutic efficacy of the vaccine elicited T cells is often hampered by the strong immune-suppressive micro-environment present at the tumor site. Since CTLA-4 antibodies can block co-inhibitory signals during T cell priming and can interfere with the immune-suppressive functions of regulatory T cells, we assessed whether combining TriMix based mRNA vaccination with anti-CTLA4 antibodies would confer increased antitumor efficacy.

We have now surprisingly found that although anti-CTLA4 monotherapy had no therapeutic effect whatsoever, the combination of Trimix based mRNA vaccination with anti-CTLA4 therapy shows superior anti-tumor efficacy, which even outperforms Trimix monotherapy.

### SUMMARY OF THE INVENTION

The present invention is defined by the following numbered statements:

1. A combination comprising:
one or more mRNA molecules encoding at least one functional immunostimulatory protein selected from the list comprising: CD40L, CD70 and caTLR4; and
a CTLA4 pathway inhibitor which prevents or blocks CTLA4 initiated signalling.
   2. The combination as defined in statement 1; wherein said one or more mRNA molecules encode all of the functional immunostimulatory proteins selected from the list comprising: CD40L, CD70 and caTLR4.
   3. The combination as defined in statement 1; wherein said CTLA4 pathway inhibitor is in the form of mRNA encoding said CTLA4 pathway inhibitor.
   4. The combination as defined in statement 1; wherein said CTLA4 pathway inhibitor is an antagonistic antibody, nanobody or derivative thereof, directed against CTLA4.
   5. The combination as defined in statement 4; wherein said antagonistic antibody directed against CTLA4 is selected from the list comprising: ipilimumab and tremelimumab.
   6. The combination as defined in anyone of statements 1-5; further comprising one or more mRNA molecules encoding a tumor-specific antigen.
   7. The combination as defined in anyone of statements 1-6; wherein said one or more mRNA molecules are formulated for parenteral administration; more in particular for intravenous, intratumoral, intradermal, subcutaneous, intraperitoneal, intramuscular or intranodal administration.
   8. The combination as defined in anyone of statements 1-7; wherein said mRNA molecules are encompassed in nanoparticles.
   9. The combination as defined in anyone of statements 1-8; wherein said nanoparticles are selected from the list comprising: lipid nanoparticles and polymeric nanoparticles.
   10. The combination as defined in anyone of statements 1-7; wherein said mRNA molecules are formulated for intranodal or intratumoral administration, and are in the form of naked mRNA molecules in a suitable injection buffer, such as a Ringer Lactate buffer.
   11. The combination as defined in anyone of statements 1-10; wherein said CTLA4 pathway inhibitor is formulated for parenteral administration; more in particular for intravenous, intratumoral, intradermal, subcutaneous, intraperitoneal, intramuscular or intranodal administration.
   12. A vaccine comprising the combination as defined in anyone of statements 1-11.
   13. The combination as defined in anyone of statements 1-11 or the vaccine as defined in statement 12 for use in human or veterinary medicine.
   14. The combination as defined in anyone of statements 1-11 or the vaccine as defined in statement 12 for use in the prevention and/or treatment of cell proliferative disorders.
   15. The combination as defined in anyone of statements 1-11 or the vaccine as defined in statement 12 for use in eliciting an immune response towards a tumor in a subject.

Hence, in a first aspect, the present invention provides a combination comprising:
one or more mRNA molecules encoding at least one functional immunostimulatory protein selected from the list comprising: CD40L, CD70 and caTLR4; and
a CTLA4 pathway inhibitor which prevents or blocks CTLA4 initiated signalling.

In a particular embodiment of the present invention, said one or more mRNA molecules encode all of the functional immunostimulatory proteins selected from the list comprising: CD40L, CD70 and caTLR4

In yet a further embodiment of the present invention, said CTLA4 pathway inhibitor is in the form of mRNA encoding said CTLA4 pathway inhibitor. Alternatively said CTLA4 pathway inhibitor is an antagonistic antibody, nanobody or derivative thereof, directed against CTLA4; more specifically said antagonistic antibody directed against CTLA4 may be selected from the list comprising: ipilumimab and tremelimumab.

In a particular embodiment, the combination of the present invention may further comprising one or more mRNA molecules encoding a tumor antigen.

In a further embodiment of the present invention, said one or more mRNA molecules are formulated for parenteral administration; more in particular for intravenous, intratumoral, intradermal, subcutaneous, intraperitoneal, intramuscular or intranodal administration.

In a particularly preferred embodiment, said combination or mRNA molecules of the present invention are formulated in nanoparticles, such as for example in lipid nanoparticles or polymeric nanoparticles.

In yet a further specific embodiment, said mRNA molecules are formulated for intranodal or intratumoral administration, and are in the form of naked mRNA molecules in a suitable injection buffer, such as a Ringer Lactate buffer.

The present invention also provides a combination as defined herein, wherein said CTLA4 pathway inhibitor is formulated for parenteral administration; more in particular for intravenous, intratumoral, intradermal, subcutaneous, intraperitoneal, intramuscular or intranodal administration.

In a further aspect, the present invention provides a vaccine comprising a combination as defined herein.

In a particular embodiment, the present invention provides a combination or vaccine as defined herein, for use in human or veterinary medicine; more in particular for use in the prevention and/or treatment of cell proliferative disorders; such as for use in eliciting an immune response towards a tumor in a subject.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Tumor growth curves of individual mice after treatment of TC-1 bearing mice with respectively PBS, anti-CTLA4, E7-TriMix mRNA or the combination of E7-TriMix mRNA with anti-CTLA-4. CR=complete responders, meaning the number of mice being tumor-free at day 90 post tumor inoculation.

FIG. 2: Mean tumor growth curves of TC-1 inoculated mice treated with PBS, anti-CTLA4, E7+TriMix mRNA and E7+TriMix mRNA+anti-CTLA4. **p=0.0012 Two-way ANOVA with Bonferroni's multiple comparisons test.

### DETAILED DESCRIPTION OF THE INVENTION

As already detailed herein above, the present invention relates to a combination comprising:
one or more mRNA molecules encoding at least one functional immunostimulatory protein selected from the list comprising: CD40L, CD70 and caTLR4; and
a CTLA4 pathway inhibitor which prevents or blocks CTLA4 initiated signalling.

Throughout the invention, the term “TriMix” stands for a mixture of mRNA molecules encoding CD40L, CD70 and caTLR4 immunostimulatory proteins. The use of the combination of CD40L and caTLR4 generates mature, cytokine/chemokine secreting DCs, as has been shown for CD40 and TLR4 ligation through addition of soluble CD40L and LPS. The introduction of CD70 into the DCs provides a co-stimulatory signal to CD27⁺ naive T-cells by inhibiting activated T-cell apoptosis and by supporting T-cell proliferation. As an alternative to caTLR4, other Toll-Like Receptors (TLR) could be used. For each TLR, a constitutive active form is known, and could possibly be introduced into the DCs in order to elicit a host immune response. In our view however, caTLR4 is the most potent activating molecule and is therefore preferred.

The mRNA or DNA used or mentioned herein can either be naked mRNA or DNA, or protected mRNA or DNA. Protection of DNA or mRNA increases its stability, yet preserving the ability to use the mRNA or DNA for vaccination purposes. Non-limiting examples of protection of both mRNA and DNA can be: liposome-encapsulation, protamine-protection, (Cationic) Lipid Lipoplexation, lipidic, cationic or polycationic compositions, Mannosylated Lipoplexation, Bubble Liposomation, Polyethylenimine (PEI) protection, liposome-loaded microbubble protection etc. In a specific embodiment, the mRNA or DNA molecules as defined herein are isolated mRNA or DNA molecules, specifically, they are preferably not part of cells (such as dendritic cells). The invention is particular intended for in vivo applications, including the direct use of isolated mRNA or DNA molecules, in contrast to ex vivo approaches encompassing the use of dendritic cells transfected with such mRNA or DNA molecules.

While the present invention is particularly suitable for use in connection with tumor-specific antigens, it may also be suitably used in connection with other types of target-specific antigens.

The term “target” used throughout the description is not limited to the specific examples that may be described herein. Any infectious agent such as a virus, a bacterium or a fungus may be targeted. In addition any tumor or cancer cell may be targeted. The term “target-specific antigen” used throughout the description is not limited to the specific examples that may be described herein. It will be clear to the skilled person that the invention is related to the induction of immunostimulation in APCs, regardless of the target-specific antigen that is presented. The antigen that is to be presented will depend on the type of target to which one intends to elicit an immune response in a subject. Typical examples of target-specific antigens are expressed or secreted markers that are specific to tumor, bacterial and fungal cells or to specific viral proteins or viral structures. Without wanting to limit the scope of protection of the invention, some examples of possible markers are listed below.

The terms “neoplasms”, “cancer” and/or “tumor” used throughout the description are not intended to be limited to the types of cancer or tumors that may have been exemplified. The term therefore encompasses all proliferative disorders such as neoplasma, dysplasia, premalignant or precancerous lesions, abnormal cell growths, benign tumors, malignant tumors, cancer or metastasis, wherein the cancer may be selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma. Specific antigens for cancer can e.g. be MelanA/MART1, Cancer-germline antigens, gp100, Tyrosinase, CEA, PSA, Her-2/neu, survivin, telomerase.

In a preferred embodiment of the vaccine of the invention, the mRNA or DNA molecule(s) encode(s) the CD40L and CD70 immunostimulatory proteins. In a particularly preferred embodiment of the vaccine of the invention, the mRNA or DNA molecule(s) encode(s) CD40L, CD70, and caTLR4 immunostimulatory proteins.

Said mRNA or DNA molecules encoding the immunostimulatory proteins can be part of a single mRNA or DNA molecule. Preferably, said single mRNA or DNA molecule is capable of expressing the two or more proteins simultaneously. In one embodiment, the mRNA or DNA molecules encoding the immunostimulatory proteins are separated in the single mRNA or DNA molecule by an internal ribosomal entry site (IRES) or a self-cleaving 2a peptide encoding sequence.

In a specific embodiment, one or more of said mRNA molecules of the present invention may further contain a translation enhancer and/or a nuclear retention element. Suitable translation enhancers and nuclear retention elements are those described in WO2015071295.

Cytotoxic T lymphocyte antigen-4 (CTLA-4) is mainly expressed in the intracellular compartment of T cells. Upon activation of a naive T cell, CTLA-4 is transported to the cell surface and concentrated at the immunological synapse, where it competes with CD28 for CD80/CD86 and down-modulates TCR signaling.

As used herein, the term “CTLA4 pathway inhibitor” includes any compound which prevents or blocks CTLA4 initiated signaling. It may thus directly or indirectly affect the regulation of CTLA4 by reducing for example the expression of the CTLA4 receptor (i.e., transcription and/or the translation) or its natural ligands B7-1 (CD80) and B7-2 (CD86). Without being so limited, such inhibitors include siRNA, antisense molecules, proteins, peptides, small molecules, antibodies, nanobodies and derivatives of any of these. In a particular embodiment, said CTLA4 inhibitor may also be provided in the form of mRNA encoding said inhibitor, such as mRNA encoding an anti-CTLA4 antibody.

The preferred anti-CTLA4 antibody is a human antibody that specifically binds to human CTLA4. Human antibodies provide a substantial advantage in the treatment methods of the present invention, as they are expected to minimize the immunogenic and allergic responses that are associated with use of non-human antibodies in human patients.

Exemplary human anti-CTLA4 antibodies are described in detail in for example WO 00/37504. Such antibodies include, but are not limited to, 3.1.1, 4.1.1, 4.8.1, 4.10.2, 4.13.1, 4.14.3, 6.1.1, ticilimumab, 11.6.1, 11.7.1, 12.3.1.1, and 12.9.1.1, as well as tremelimumab and ipilimumab. In another embodiment, the antibody is selected from an antibody having the full length, variable region, or CDR, amino acid sequences of the heavy and light chains of the above-defined antibodies.

In other embodiments of the invention, the antibody inhibits binding between CTLA4 and B7-1, B7-2, or both. Preferably, the antibody can inhibit binding with B7-1 with an IC₅₀ of about 100 nM or lower, more preferably, about 10 nM or lower, for example about 5 nM or lower, yet more preferably, about 2 nM or lower, or even more preferably, for example, about 1 nM or lower. Likewise, the antibody can inhibit binding with B7-2 with an IC₅₀ of about 100 nM or lower, more preferably, 10 nM or lower, for example, even more preferably, about 5 nM or lower, yet more preferably, about 2 nM or lower, or even more preferably, about 1 nM or lower.

While the anti-CTLA4 antibodies discussed previously herein may be preferred, the skilled artisan, based upon the disclosure provided herein, would appreciate that the invention encompasses a wide variety of anti-CTLA4 antibodies and is not limited to these particular antibodies. More particularly, while human antibodies are preferred, the invention is in no way limited to human antibodies; rather, the invention encompasses useful antibodies regardless of species origin, and includes, among others, chimeric humanized and/or primatized antibodies.

In one embodiment, the disclosure provides a method for a combination therapy for individuals comprising administering to the individual one or more immunostimulatory factors and an inhibitor of a CTLA4 pathway. The one or more immunostimulatory factors and the CTLA4 pathway inhibitor may be administered simultaneously or contemporaneously, as a single composition or separate compositions, or the one or more immunostimulatory factors and CTLA4 pathway inhibitor may be administered at different times.

The compositions of the present invention generally include a CTLA4 pathway inhibitor in combination with one or more immunostimulatory factors.

Suitable dosages of the CTLA4 pathway inhibitor will depend upon a number of factors including, for example, age and weight of an individual, at least one precise condition requiring treatment, severity of a condition, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

The present invention also provides a combination as defined herein; wherein said mRNA molecules are encompassed in nanoparticles.

As used herein, the term “nanoparticle” refers to any particle having a diameter making the particle suitable for systemic, in particular intravenous administration, of, in particular, nucleic acids, typically having a diameter of less than 1000 nanometers (nm).

In a specific embodiment of the present invention, the nanoparticles are selected from the list comprising: lipid nanoparticles and polymeric nanoparticles.

A lipid nanoparticle (LNP) is generally known as a nanosized particle composed of a combination of different lipids. While many different types of lipids may be included in such LNP, the LNP's of the present invention may for example be composed of a combination of an ionisable lipid, a phospholipid, a sterol and a PEG lipid.

A polymeric nanoparticle can typically be a nanosphere or a nanocapsule. Two main strategies are used for the preparation of polymeric nanoparticles, i.e. the “top-down” approach and the “bottom-up” approach. In the top-down approach a dispersion of preformed polymers produces polymeric nanoparticles, whereas in the bottom-up approach, polymerization of monomers leads to the formation of polymeric nanoparticles. Both top-down and bottom-up methods use synthetic polymers/monomers like poly(d, I-lactide-co-glycolide), poly(ethyl cyanoacrylate), poly(butyl cyanoacrylate), poly(isobutyl cyanoacrylate), and poly(isohexyl cyanoacrylate); stabilizers like poly(vinyl alcohol) and didecyldimethylammonium bromide; and organic solvents like dichloromethane and ethyl acetate, benzyl alcohol, cyclohexane, acetonitrile, acetone, and so on. Recently the scientific community has been trying to find alternatives for synthetic polymers by using natural polymers and synthesis methods with less toxic solvents.

The present invention also provides the combinations and vaccines as defined herein for use in human or veterinary medicine, in particular for use in the treatment of cell proliferative disorders, more in particular for use in eliciting an immune response towards a tumor in a subject.

Finally, the present invention provides a method for the treatment of a cell proliferative disorder comprising the steps of administering to a subject in need thereof a combination or vaccine of the present invention.

The compositions may also be of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal.

The invention will now be illustrated by means of the following synthetic and biological examples, which do not limit the scope of the invention in any way.

### EXAMPLES

### Example 1: Antitumor Efficacy of Trimix Based Immunization as Monotherapy or in Combination with Anti-CTLA4 Antibodies in Mice Bearing Tc-1 Tumors

### Materials and Methods:

Synthesis of In Vitro Transcribed mRNA:

E7 mRNA and mouse caTLR4, mouse CD70 and mouse CD40L (TriMix) mRNA were synthesized from the corresponding linearized peTheRNA plasmids by in vitro transcription as previously described (EP3068888).

### Mice:

C57BL/6 mice (female, 6 wks old) were purchased from Janvier (Genest) and maintained under SPF (OncoDesign, Dijon) conditions according to FELASA guidelines. TC-1 cells were obtained from ATCC and cultured as described previously.

### Antibodies:

Anti-CTLA4 antibody was purchased from BioXCell (ref: BE0131; clone: 9H10; reactivity: mouse; isotype: Hamster IgG1; storage conditions: +4° C.).

### Tumor Inoculation and Treatment Schedule:

At day 0, mice were subcutaneously inoculated with 1×10⁶ TC-1 tumor cells in a volume of 200 μl of PBS. Intranodal immunizations with E7/TriMix mRNA were performed at days 3, 8 and 13 post tumor inoculation. Mice received 10 μg of E7 mRNA combined with 30 μg of TriMix mRNA dissolved in 0,8×Ringer's Lactate solution (20 μl). mRNAs were injected into the inguinal lymph node. Anti-CTLA4 antibody was injected intraperitoneally (10 mg/kg/administration) at days 3, 6, 9 and 12 post tumor inoculation.

### Intranodal Administrations

Mice were shaved on the inguinal region to remove fur prior to disinfection with 70% ethanol. A small incision was made in the inguinal area to expose the inguinal lymph node. A total volume of 10 μl of mRNA solution was injected into the inguinal lymph node using a 0.3 ml 30G insulin needle (BD Biosciences, Ref 324826A). Following injection, skin was closed with 4-0 crinerce sutures. The same inguinal lymph node was injected for all three intranodal immunizations.

### Results:

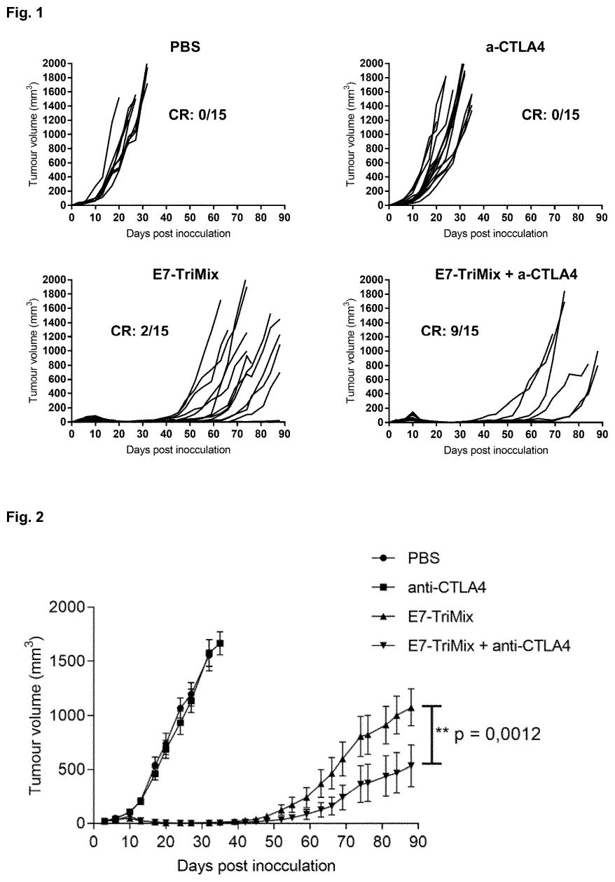

As detailed in FIGS. 1 and 2, the described regimen of anti-CTLA-4 monotherapy does not provide any therapeutic benefit whatsoever in the TC-1 model compared to the controls (PBS), with no complete responders in either group. On the other hand, intranodal immunization with E7/TriMix was shown to strongly delay TC-1 tumor growth and results in a limited fraction of complete responders (2/15).

Even more striking is that the combination of anti-CTLA4 antibodies with intranodal E7/TriMix immunization shows superior anti-tumor efficacy compared to both intranodal E7/TriMix monotherapy (p=0.0012) and anti-CTLA4 monotherapy and strongly increases the fraction of complete responders (9/15).

Hence, these data clearly show the potential of the combination of Trimix and CTLA4 pathway inhibitory molecules in tumor therapy.

### REFERENCES

Bonehill A et al. Mol. Ther. 2008; 16:1170-80.
Kariko K et. Immunity 2005; 23, 165-175.
Sahin et al. Nat. Discovery Rev. 2014; 13, 759-780.
Seidel et al. Front Oncol. 2018; 8: 86.
Van Lint S. et al. Cancer Res. 2012; 1; 72(7):1661-71
Van Lint S. et al. Cancer Res Immunol. 2016; 4 (2):146-156
Yoneyama, M. & Fujita, T. Rev. Med. Virol. 2010; 20, 4-22

1-15. (canceled)

16. A combination comprising:
one or more isolated mRNA molecules encoding at least one functional immunostimulatory protein selected from CD40L, CD70, and caTLR4; and
a CTLA4 pathway inhibitor that prevents or blocks CTLA4 initiated signalling.

17. The combination of claim 16, wherein the one or more mRNA molecules encode CD40L, CD70, and caTLR4

18. The combination of claim 16, wherein the CTLA4 pathway inhibitor is in the form of mRNA encoding the CTLA4 pathway inhibitor.

19. The combination of claim 16, wherein the CTLA4 pathway inhibitor is an antagonistic antibody directed against CTLA4, a nanobody directed against CTLA4, or a derivative of the anatagonistic antibody or the nanobody.

20. The combination of claim 19, wherein the antagonistic antibody directed against CTLA4 is ipilumimab or tremelimumab.

21. The combination of claim 16, further comprising one or more mRNA molecules encoding a tumor antigen.

22. The combination of claim 16, wherein the one or more mRNA molecules are formulated for parenteral administration.

23. The combination of claim 16, wherein the one or more mRNA molecules are formulated for intravenous administration, intratumoral administration, intradermal administration, subcutaneous administration, intraperitoneal administration, intramuscular administration or intranodal administration.

24. The combination of claim 16, wherein the mRNA molecules are formulated in nanoparticles.

25. The combination of claim 24, wherein the nanoparticles are selected from the group consisting of lipid nanoparticles and polymeric nanoparticles.

26. The combination of claim 16, wherein:
the mRNA molecules are formulated for intranodal or intratumoral administration; and
the mRNA molecules are in the form of naked mRNA molecules in an injection buffer.

27. The combination of claim 16, wherein the CTLA4 pathway inhibitor is formulated for parenteral administration

28. The combination of claim 16, wherein the CTLA4 pathway inhibitor is formulated for intravenous administration, intratumoral administration, intradermal administration, subcutaneous administration, intraperitoneal administration, intramuscular administration or intranodal administration.

29. The combination of claim 16, wherein the combination does not comprise cells.

30. The combination of claim 16, wherein the combination does not comprise dendritic cells.

31. A method for eliciting an immune response toward a tumor in a subject, the method comprising:
administering to the subject the combination according to claim 16.

32. A method for treating a cell proliferative disorder in a subject having the cell proliferative disorder, the method comprising:
administering to the subject the combination according to claim 16.

33. A vaccine comprising the combination according to claim 16.

34. A method for treating a cell proliferative disorder in a human or veterinary subject having the cell proliferative disorder, the method comprising:
administering to the human or veterinary subject the vaccine according to claim 33.

35. A method for eliciting an immune response toward a tumor in a subject, the method comprising:
administering to the subject the vaccine according to claim 33.
